(19) United States
(12) Patent Application Publication
(10) Pub. No.: US 2021/0322266 A1
(43) Pub. Date: Oct. 21, 2021

(54) PORTABLE HYDROTHERAPY APPARATUS

(71) Applicant: Ciara Brooks, Spring, TX (US)

(72) Inventor: Ciara Brooks, Spring, TX (US)

(21) Appl. No.: 17/216,672

(22) Filed: Mar. 30, 2021

Related U.S. Application Data
(60) Provisional application No. 63/010,387, filed on Apr. 15, 2020.

(51) Int. Cl. A61H 33/00; A61N 5/06; A61H 33/02

(52) U.S. Cl. CPC A61H 33/6068; A61H 33/6063; A61N 5/0613; A61H 33/027; A61H 2201/013; A61N 2005/0651; A61N 2005/0663; A61H 2201/50; A61H 2201/1207; A61H 2201/10

(57) ABSTRACT

This invention relates to a portable bath and hydrotherapy apparatus, utilizing an electric or a battery-operated motor. More particularly, the invention relates to a portable hydrotherapy unit which can be used in conjunction with a bathtub or with other open-topped bodies adapted to be partially filled with a supply of liquid. The improved resultant of the present invention is the utilization of such a motor for hydrotherapy purposes whereby the unit re-circulates the water of the bathtub.

### CROSS-REFERENCE TO RELATED APPLICATION

The present invention claims priority to provisional application Ser. No. 63/010,387, filed Apr. 15, 2020 and entitled “PORTABLE HYDROTHERAPY APPARATUS” is hereby incorporated in its entirety at least by reference.

### BACKGROUND

### 1. Field of the Invention

The present invention relates to a portable hydrotherapy apparatus for providing hydrotherapy within a bathtub.

### 2. Description of Related. Art

Hydrotherapy is the use of water in the treatment of different conditions, including cerebral palsy (CP), arthritis, and rheumatic conditions. It also soothes sore or inflamed muscles and joints, rejuvenates injured limbs, lowers fevers, soothes headaches, promotes relaxation, treats burns and frostbite, eases labor pains, and eases up skin problems.

Hydrotherapy is often undertaken at health centers, spas, or at home. Usually, the conventional commercial hydrotherapy units are too expensive and too bulky to be of substantial use in an average household. The present invention solves the problem of using an expensive hydrotherapy apparatus at home by a relatively inexpensive portable hydrotherapy device, which can be used to convert a bathtub into a hydrotherapy apparatus. Moreover, the present invention allows scope of re-circulation of water in a bath-tub, optimizing water consumption and at the same time the objectives of the hydrotherapy apparatus. Efforts focusing on the above objective found a few novel ideas in vogue.

An US patent publication no US20130185864A1 and titled “Multifunctional spa device” discusses about a multifunctional spa device. It is mainly that an inlet of a pump controlled by a circuit is additionally connected to a vent pipe, an outlet of the pump is connected to a jet, and the pump sucks a water flow through an intake pipe while air is simultaneously sucked through the vent pipe to be mixed with the water flow in the pump. An outlet disk is configured in the jet, and pores are distributed on an outer circumference of the outlet disk. With the jet inside-out to transmit the air-mixing water flow, the air-mixing water flow is further compressed and released through the pores, thus to output water flows or the water columns carried with ultra-micro bubbles.

Another U.S. Pat. No. 2,591,252A titled “Portable hydrotherapy apparatus” discusses about a hydrotherapy apparatus which includes a tank, a liquid inlet conduit which enters said tank through the lower part thereof having a vertically extending portion within said tank, a pump external of said tank for supplying liquid under pressure to said conduit, and an aeration injection unit movably supported within said tank having a conduit in telescoping relation with said vertically extending portion.

Another U.S. Pat. No. 3,911,505A titled “Hydrotherapy apparatus” discusses about a hydrotherapy apparatus consisting of a portable unit which can be removably mounted in a bathtub or the like. The immersible portion of the unit contains pumping means and water conditioning means. Water from the bath flows into the immersible portion where it is heated or cooled and from there discharged into the bath. The non-immersible portion of the unit contains the necessary controls.

However, none of the above-mentioned prior art tallies in totality with the present application especially the re-circulation of water in a bathtub, keeping all the health benefits of hydrotherapy intact.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

It is an object of the present invention to provide a hydrotherapy apparatus adapted to be partly immersed beneath the surfaces of a body of liquid contained in an open-topped body adapted to be partially filled with a supply of liquid.

It is another object of the present invention to provide a compactly designed, light weight and portable hydrotherapy apparatus comprising of relatively few parts, with a minimum of moving parts, thereby creating a unit which can be easily disassembled.

It is another object of the present invention to provide a simple and inexpensive hydrotherapy unit which can be removably mounted on a bathtub or the like.

In order to do so, a portable hydrotherapy apparatus for hydrotherapy within a bathtub, the apparatus comprising a housing containing a motor and a battery; a fluid inlet conduit having an inlet, the fluid inlet conduit in fluid communication with the housing and a water supply configured to be retained in the bathtub during use, a fluid outlet conduit having an outlet positioned on an inner surface of the bathtub, the fluid inlet conduit in fluid communication with the fluid inlet conduit via the housing, wherein the housing is configured to permit water from the water supply to enter the fluid inlet conduit and exit the fluid outlet conduit; and, a water attachment coupled to the outlet, the water attachment configured to project a varying volume of water outwardly.

In one embodiment, a plurality of fluid outlet conduits in conjunction with a plurality of water attachments is provided. In one embodiment, the inlet is mounted within the inner surface of the bathtub approximate to a bottom surface of the bathtub. In one embodiment, the water attachments include LED lights providing color therapy. In another embodiment, the LED lights may be any one of a pinkish purplish color, a sunset color, a blue color, or a green color. In one embodiment, a remote control configured to control the motor and settings of the water attachment including the varying volume of water is provided. In one embodiment, a volume of air is mixed with the volume of water. In one embodiment, the volume of air or the volume of water is projected at a varying frequency or intervals.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### BRIEF DESCRIPTION OF THE SEVERAL VIEWS OF TI-IE DRAWING(S

The detailed description of some embodiments of the invention is made below with reference to the accompanying figures, wherein like numerals represent corresponding parts of the figures.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is a perspective view of a portable hydrotherapy apparatus mounted on a bathtub according to an embodiment of the present invention; and,

FIG. 2 is a top view of the portable hydrotherapy apparatus mounted on a bathtub according to an embodiment of the present invention.

### DETAILED DESCRIPTION OF CERTAIN EMBODIMENTS

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

As used in this application, the term. “a” or “an” means “at least one” or “one or more.” As used in this application, the term “about” or “approximately” refers to a range of values within plus or minus 10% of the specified number. As used herein, the term “about” refers to an amount that is near the stated amount by about 0%, 5%, or 10%, including increments therein. As used in this application, the term “substantially” means that the actual value is within about 10% of the actual desired value, particularly within about 5% of the actual desired value and especially within about 1% of the actual desired value of any variable, element or limit set forth herein. Unless otherwise defined, all technical terms used herein have the same meaning as commonly understood by one of ordinary skill in the art to which this disclosure belongs.

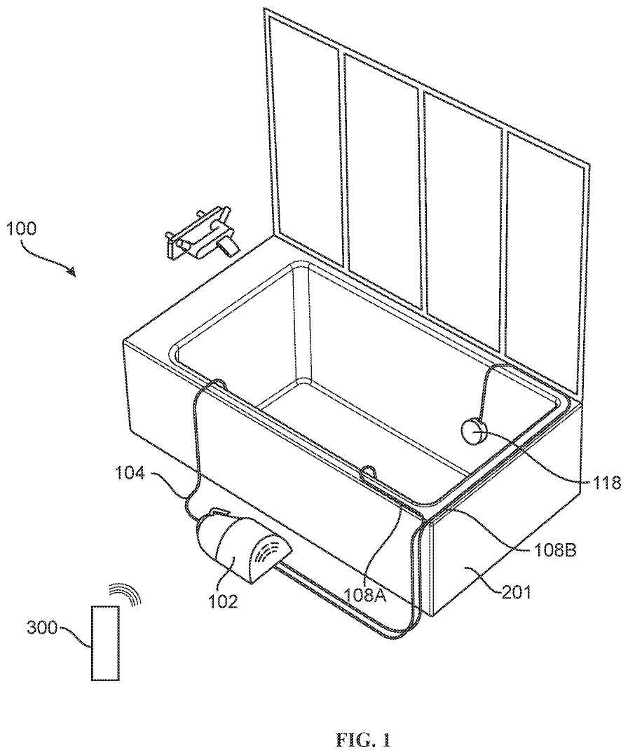

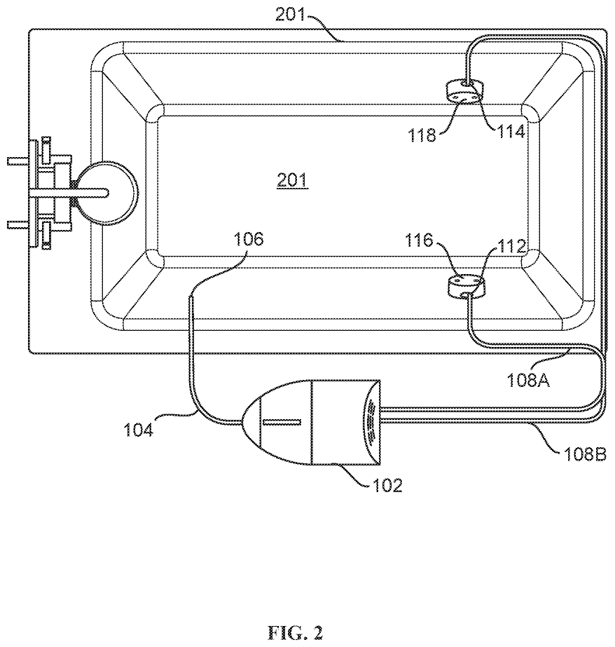

Referring now to FIGS. 1-2, the portable hydrotherapy apparatus 100 is mounted on a bathtub 201 in one embodiment of the present invention. In some embodiments, the portable hydrotherapy apparatus comprises a housing 102 containing a motor and battery within. Although not shown, the motor and battery are any known type of the prior art. In some embodiments, the housing 102 is coupled with a fluid inlet conduit 104, the fluid inlet conduit 104 allowing fluid from a water source to travel to the housing via inlet 106. In some embodiments, the water source is the water present in the bathtub 201 during use.

In some embodiments, the inlet 106 is approximate to a bottom 201 surface of the bathtub 203. In some embodiments, at least one fluid outlet conduit 108 is provided, in the embodiment shown there is a pair of fluid outlet conduits 108A and 108B. The fluid outlets are configured to enable water from the housing 102 to travel through the conduits 108A and 108B to outlets 112 and 114 respectively. In some embodiments, water attachments 116 and 118 are in fluid communication with conduits 108A and 108B. The waters attachments provide a means for projecting the water outward via the conduits, including but not limited to jet, pulse, waves, vibration, mist, wherein the water projection is any volume of water-air ratio at one or varying frequencies depending on the motor settings. As previously indicated, the type of motor may vary.

In some embodiments, the water attachments are secured to the wall of the bath tub by means of suction cups (not shown). The water attachments may be positioned at varying locations on the bathtub.

In one embodiment, battery is a rechargeable battery, configured to power the jet outflow. For controlling the jet outflow, multiple settings are provided, for example, in the jet flow can be adjusted at 20%, 60% or 100% intensity water outflow, depending upon the requirement of the individual. In some embodiments, a remote control 300 may be used with the apparatus for controlling the motor and/or the water attachment settings.

In some embodiments, LED lights are provided on the water attachments, the LED lights providing color therapy. In some embodiments, the LED lights may be any one of pinkish purplish color, a sunset (red, orange, yellow) color, blue color, green color, or combination thereof. It should be understood that the LED lighting is optional.

The length and position of any conduit may be adjusted for the individual space or location it is used in conjunction with, i.e. the bath tub. Likewise, the diameter and material of any conduit may vary.

In operation, the inlet 106 is immersed in a bathtub. Once the motor is activated by either switching it on remotely or by an adequately designed on-off switch manually and the bathtub is filled with water to the desired level, the motor begins to operate and withdraw water through the inlet, into the fluid inlet conduit 104 and the water is then forced out through the outlets 108A and/or 108B into the bathtub. In some embodiments, the water can be mixed with air as previously described. The water attachments are installed at a height suitable for providing message to the user.

Although the invention has been described in considerable detail in language specific to structural features, it is to be understood that the invention defined iii the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternate embodiments are contemplated, and can be made without departing from the spirit and scope of the invention. For example, in some embodiments, the fluid via the conduits is air, without water.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

Unless otherwise defined, all technical terms used herein have the same meaning as commonly understood by one of ordinary skill in the art to which this disclosure belongs.

Any element in a claim that does not explicitly state “means for” performing a specified function, or “step for” performing a specified function, is not to be interpreted as a “means” or “step” clause as specified in 35 U.S.C. § 1.12, ¶6. In particular, any use of “step of” in the claims is not intended to invoke the provision of 35 U.S.C. § 112, ¶ 6.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A portable hydrotherapy apparatus for hydrotherapy within a bathtub, the apparatus comprising:
a housing containing a motor and a battery;
a fluid inlet conduit having an inlet, the fluid inlet conduit in fluid communication with the housing and a water supply configured to be retained in the bathtub during use,
a fluid outlet conduit having an outlet positioned on an inner surface of the bathtub, the fluid inlet conduit in fluid communication with the fluid inlet conduit via the housing, wherein the housing is configured to permit water from the water supply to enter the fluid inlet conduit and exit the fluid outlet conduit; and,
a water attachment coupled to the outlet, the water attachment configured to project a varying volume of water outwardly.

2. The portable hydrotherapy apparatus of claim 1, further comprising a plurality of fluid outlet conduits in conjunction with a plurality of water attachments.

3. The portable hydrotherapy apparatus of claim 1, wherein the inlet is mounted within the inner surface of the bathtub approximate to a bottom surface of the bathtub.

4. The portable hydrotherapy apparatus of claim 1, wherein the water attachment include LED lights providing color therapy.

5. The portable hydrotherapy apparatus of claim 4, wherein the LED lights may be any one of a pinkish purplish color, a sunset color, a blue color, or a green color.

6. The portable hydrotherapy apparatus of claim 1, further comprising a remote control configured to control the motor and settings of the water attachment including the varying volume of water.

7. The portable hydrotherapy apparatus of claim 1, wherein a volume of air is mixed with the volume of water.

8. The portable hydrotherapy apparatus of claim 7, wherein the volume of air or the volume of water is projected at a varying frequency or intervals.
